(19) United States
(12) Patent Application Publication
(10) Pub. No.: US 2007/0073366 A1
(43) Pub. Date: Mar. 29, 2007

(54) Portable rechargeable therapeutic device and method of using the same

(75) Inventor: Gino M. Porco, Toronto (CA)

Correspondence Address: LANG MICHENER, BCE PLACE, P.O. BOX 747, SUITE 2500, 181 BAY STREET, TORONTO, ON, M5J 2T7, CA

(73) Assignee: Infinity Brands Inc.

(21) Appl. No.: 11/408,989

(22) Filed: Apr. 24, 2006

(30) Foreign Application Priority Data
Apr. 22, 2005 (CA) 2,504,857

(51) Int. Cl. A61N 5/06; A61H 1/00; A61H 1/02; A61H 5/00

(52) U.S. Cl. 607/89; 601/15

(57) ABSTRACT

A hand-held, portable, rechargeable, light-weight, personal therapeutic device that uses photo-biostimulation for personal home therapy treatments is disclosed. The device combines Low Level Laser Therapy (LLLT), which produces coherent radiation, with incoherent radiation from light emitting diodes (LED), and stimulating massage. The device permits the user selection of one or more of the therapeutic treatments as appropriate for the ailment being treated and a method of using the apparatus is also disclosed.

### FIELD OF THE INVENTION

This invention relates to the field of medical and therapeutic treatments, and in particular to a portable, rechargeable device for personal therapeutic application of one or more of coherent light treatment, incoherent light treatment and massage treatment.

### BACKGROUND OF THE INVENTION

Laser (Light Amplification by Stimulated Emission of Radiation) was first theorized by Einstein. In 1960, Miamian developed the first laser, a ruby laser. This was a tube laser with a metal chamber, which contained a ruby element. When an electrical current excited the enclosed element, the atoms give off photons or packets of light energy. The photons bounced off a solid mirror on one end of the tube and out holes in the mirror on the other end of the tube. This light beam is unlike regular light in that it is coherent i.e., the photons are well ordered and synchronized. Laser light is also monochromatic, meaning it is of one pure color.

Power density is a key to laser energy. Power density (PD), or light concentration is measured in watts per centimeter squared (W/cm²). The problem with most DC battery driven lasers is that the battery bleeds off and does not maintain a standard PD, which negatively affects low-level laser therapy (LLLT) results. Recent advances in miniature computers have enabled the development of techniques that maintain a standard PD as well as to control energy frequency.

Wavelengths are measured in nanometers. The most therapeutically beneficial wavelengths are in the visible and near infrared ranges. These ranges are very safe ranges, far away from the damaging ultraviolet, x-rays, gamma and cosmic rays. Although the longer waves such as microwaves and radio waves are usually considered safe, there are some that think they might be damaging to the very sensitive individual.

All wavelengths used in low-level lasers are safely divided from these potentially damaging waves.

Many people only think of lasers as cutting lasers. In order to cut with lasers, it is necessary to increase the PD from 300 to 10,000 W/cm². Lasers do not even have a warming affect unless they are operated above 5 W/cm².

Low-level lasers today are manufactured using semi-conductors grown from various pure elements or combinations thereof Combining the elements of InGaAlP makes visible light in the range of 630 to 685 nm; combining GaAlAs produces light in the range of 780 to 870 nm; and, combining GaAs produces infrared laser diodes in the 900 nm range.

LED stands for light emitting diode. An LED is a silicon microchip with various added substances, each of which: releases a different wavelength (color) of light when electrically stimulated. LEDs used to be used mainly as low-power indicator lights for electronic devices. Now manufacturers are racing to release LEDs with higher intensities and a greater range of available colors and designs

The visible light ranges, while quite beneficial, are limited by its shallow penetration of 1 to 3 mm. The invisible or infrared light range penetrates much deeper. Research documents infrared penetrations from 10 to 15 mm, but clinical results indicate that the infrared beam penetrates 8 to 10 cm.

It has been shown that laser energy is accumulative as well as cascading and reduces pain and inflammation via: bio-stimulation and photo-stimulation; endogenous opiate production; Slowing sensory nerve production; restoring cellular resonant energy; stimulating the Na/K pump mechanism in the cell membrane; and, inhibiting bradykinin and leukotriene production.

LLLT is the application of red and near infra-red light over injuries or lesions to improve wound or soft tissue healing and give relief for both acute and chronic pain. LLLT is used to: increase the speed, quality and tensile strength of tissue repair; give pain relief; resolve inflammation; improve function of damaged neurological tissue and often used as an alternative to needles for acupuncture.

The red and near infrared light (600 nm-1000 nm) commonly used in LLLT can be produced by laser or high intensity LED. The intensity of LLLT lasers and LEDs is not high like a surgical laser. There is no heating effect.

The effects of LLLT are photochemical, like photosynthesis in plants. Red and near infrared light can affect cell membrane permeability and aid the production of ATP, thereby providing the cell with more energy which in turn means the cell is in optimum condition to play its part in a natural healing, or restorative process.

Clinically operated LLLT devices are typically delivering 10 mW 500 mW. The power density typically ranges from 0.05-5 W/cm².

LLLT can be used for soft tissue injuries; joint conditions, chronic pain, non healing wounds and ulcers, post-op pain and acupuncture, and has shown tremendous promise in topical treatment of androgenitic alopecia (male pattern baldness), as well as the treatment of skin conditions, lesions, burns and reduction of scarring.

Like photosynthesis, the correct wavelengths and power of light at certain intensities for an appropriate period of time can increase ATP production and cell membrane perturbation could lead to permeability changes and second messenger activity resulting in functional changes such as increased syntheses increased secretion and motility changes. Red and near infrared light seem to be the most ideal wavelengths.

Red light acts on the mitochondria and near infrared light on the mitochondria and at the cell membrane. Shorter wavelengths are not as good, expensive to produce and with poor penetration they are a poor choice. Near infrared light, while not quite as good, do penetrate better than the red wavelengths and are available in higher powers and at lower prices. It is understood that 810 nm is the best penetrating wavelength for deep tissue restorative treatments.

An appropriate dose of light can improve speed and quality of acute and chronic wound healing, soft tissue healing, pain relief improve the immune system and nerve regeneration. Applications with good recovery evidence include venous ulcers, diabetic ulcers, osteoarthritis, tendonitis, post herpetic neuralgia, shingles and postoperative pain.

### SUMMARY OF THE INVENTION

Low-energy photon irradiation by light in the far-red to near-IR spectral range with low-energy (LLLT) lasers or LED arrays has been found to modulate various biological processes in cell culture and animal models. This phenomenon of photobiomodulation has been applied clinically in the treatment of soft tissue injuries and the acceleration of wound healing. The mechanism of photobiomodulation by red to near-IR light at the cellular level has been ascribed to the activation of mitochondrial respiratory chain components, resulting in initiation of a signaling cascade that promotes cellular proliferation and cytoprotection.

It has been suggested that cytochrome oxidase is a key photoacceptor of light in the far-red to near-IR spectral range. Cytochrome oxidase is an integral membrane protein that contains four redox active metal centers and has a strong absorbance in the far-red to near-IR spectral range detectable in vivo by near-IR spectroscopy.

Moreover, 660-680 nm of irradiation has been shown to increase electron transfer in purified cytochrome oxidase, increase mitochondrial respiration and ATP synthesis in isolated mitochondria, and up-regulate cytochrome oxidase activity in cultured neuronal cells.

LED photostimulation induces a cascade of signaling events initiated by the initial absorption of light by cytochrome oxidase. These signaling events may include the activation of immediate early genes, transcription factors, cytochrome oxidase subunit gene expression, and a host of other enzymes and pathways related to increased oxidative metabolism.

In addition to increased oxidative metabolism, red to near-IR light stimulation of mitochondrial electron transfer is known to increase the generation of reactive oxygen species. These mitochondrially generated reactive oxygen species may function as signaling molecules to provide communication between mitochondria and the cytosol and nucleus.

It has been discovered that the combination of LLLT, LED and massage therapies have a beneficial impact for many topical applications including: chronic pain; acute (traumatic) pain; neck and back pain; tendonitis; sports injuries; painful trigger points; carpal tunnel syndrome; tennis/golfer elbow; acne, eczema; inflammatory skin conditions; wounds and burns; non-healing ulcers; rheumatoid arthritis; fibromyalgia; lymph edema; osteoarthritis; migraines; post herpetic neuralgia; scars and colloids; sinusitis, tonsillitis; TMJ disorders; hearing loss; Meniere's Disease; tinnitus; vertigo; hair loss and recovery; This treatment results in the provision of additional blood flow, nutrient delivery, and in some treatment cases, the reduction in build up of harmful or degenerative compounds at a micro-biological level.

The invention is designed for topical treatment regimens only, and thus does not produce any infrared light range. The light energy penetration of the invention is limited to only the outer layers of the epidermal and dermal skin layers. The invention combines the light energies from both sources, creating a unique treatment regimen. It uses InGaAlP lasers producing visible laser light at 650 nm. The invention is a topical therapy device, and is not designed to provide deep tissue penetration.

The invention provides light energy to assist in healing, to energize cellular metabolism, and create an easy, convenient, and entirely safe way to access the many benefits of this therapy at home.

As with most forms of energy medicine, therapeutic light is applied in a more intense manner than that which is ambient in the environment, with more precise selection of color (wavelength) that is supportive of the individual condition. A well-designed LED light therapy device is, therefore, a step between generalized bathing of the body in diffuse light and the coherent beam of a laser.

The invention combines 3 separate, but synergistic technologies to stimulate cells, and promote more rapid healing, scar reduction, wound healing, pain abatement, and skin/hair regeneration. Essentially, the invention energizes skin at a cellular level, creating a more healthy and youthful skin condition. The invention uniquely combines LLLT, LED and massage therapies in a single, hand-held, rechargeable unit.

The combination of these therapies ensures maximum stimulation to the treatment area, and a significant increase in blood flow resulting in better oxygenation of the cells, and improved trophism (blood cell nutrition), and hence better health at the cellular level.

The resulting tissue improvement can result in a more youthful appearance, healthier skin, and improved regenerative properties.

The invention is a convenient re-chargeable unit that can be used anywhere. The lightweight, hand held, cordless device is ideal for home use. The reduction or elimination of scheduled clinical visits can be viewed as a tremendous time, and cost savings by most people. Further, because the invention uses LLLT, there is no special training or clinical personnel required to administer the treatments.

In the case of the invention, the laser diodes operate at 4.5 milliwatts, providing a PD at the higher end of the very safe range, and ensuring that the invention is in fact producing “cold laser” energy, i.e. No heat is produced.

The invention is further unique due to its general application design, as opposed to being developed to combat just one condition (i.e. Hair Loss).

The invention is the first hand held home use device that combines the proven benefits of LLLT, LED Therapy, and stimulating massage to impact the vitality and health of dermal and epidermal cells. There are a wide variety of ailments that have demonstrated positive results with the application of either one, two or all three of these therapies alone or in combination.

The rechargeable nature of the invention makes it convenient to use anywhere, at home or during travel. The unit comes complete with the A/C power adapter to recharge the batteries.

Treatment timing is made easy with the invention, thanks to the automatic countdown timer. Having determined that a 15 minute treatment is the optimal treatment period, the invention automatically times each treatment session. Activation of either power button initiates the countdown timer, and upon completion of the 15 Minute treatment period, the unit will automatically shut off. This is also helpful to prevent battery run down on the device, should it be inadvertently left on.

Non-coherent light therapy is provided by the 4 LED light outputs. As has been explained, both coherent and non-coherent light energy has been proven to have therapeutic properties. The visible spectrum red, light emitted from the LED units is again provided at the optimal therapeutic value. This non-thermal, photo-chemical treatment providing light energy at a cellular level mimics the natural, non-invasive energy provided by the sun, in the light colour spectrum that NASA has deemed to be the most beneficial.

Coherent light is provided through 2 LLLT Laser Diodes at 0.4.5 Milliwatt light energy output. The laser diodes in the invention deliver near infrared light at 650 nm wavelength. Unlike other “specific” use home units, the invention does not refract or disperse the coherent light energy through the use of either lenses or refractors. This ensures the light energy delivered remains coherent and is of the quality delivered directly from the laser diode ensuring the optimal therapeutic wavelength is maintained.

The third stimulation provided by the invention, comes from the massage motor built directly into the unit. A stimulating massage can be delivered directly by the 4.8 Volt massage motor, and can be operated either by itself, or in combination with the light therapy.

Laser light energy is provided by 2 laser InGaAlP diodes, delivering 650 Nanometer (Nm)+/−5 Nm wavelength light energy. These two non-refracted, non-dispersed laser light energy sources combine to provide a therapeutic stimulation at a molecular level of the cell areas being treated. A further 4 LED outputs work in conjunction with the laser diodes to provide a combined therapy.

### BRIEF DESCRIPTION OF THE DRAWINGS

The invention will now be described in more detail, by way of example only, with reference to the accompanying drawings, in which:—

FIG. 1 is a front view of the of the device embodying the features of the present invention;

FIG. 2 a back view of the device of FIG. 1.

### DETAILED DESCRIPTION OF THE PREFERRED EMBODIMENTS

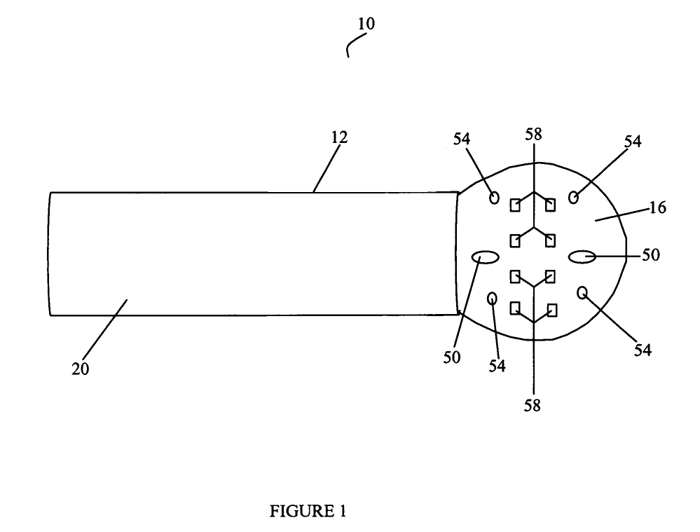

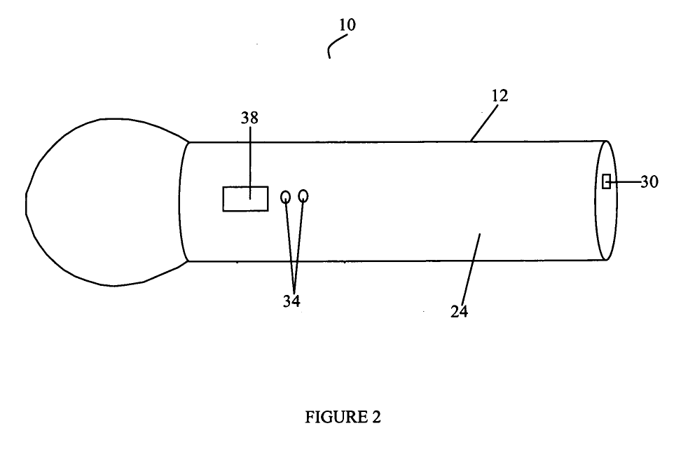

Referring now to FIG. 1, the invention in accordance with the presently preferred embodiment is generally indicated at 10. Referring now to FIGS. 1 and 2, the therapeutic device embodying features of the present invention includes a housing 12, a laser diode arrangement 50, a light emitting diode arrangement 54 and a massage element arrangement 58, all mounted on a treatment head 16. The back of the device provides an electric power source 30, either plug in use, or for recharging an internal rechargeable battery (not shown), a means for operator input 34, such as a control switch, and a means for operator output 38. Housing 12 is generally elongated, has a front 20 and a back 24, and is preferably sized and shaped to be comfortably held in the hand of an operator. Other sizes and shapes are suitable for housing 12.

The method of the present invention includes the steps of an operator activating at least one of the laser diode arrangement, the light emitting diode arrangement and the massage element arrangement and directing the device at an area of the operator's body to permit the imparting of the energy created by the device to that area.

This concludes the description of the preferred embodiment of the invention. The foregoing description has been presented for the purpose of illustration and is not intended to be exhaustive or to limit the invention to the precise form disclosed. Many modifications and variations are possible in light of the above teaching and will be apparent to those skilled in the art. It is intended the scope of the invention be limited not by this description but by the claims that follow.

We claim:

1. A therapeutic device, comprising:
a housing sized and shaped to be comfortably held in the hand of an operator, and having a front and a back;
a treatment head mounted to the front of the housing, having an arrangement of one or more laser diodes, and arrangement of one or more light emitting diodes and an arrangement of one or more massage elements therein,
a control switch mounted to the housing to permit the operator to activate any one or more of the laser diode arrangement, the light emitting diode arrangement and the massage element arrangement;
whereby the therapeutic device may be directed at an area of the operator's body to permit the imparting of the energy created by the laser diode arrangement, the light emitting diode arrangement or the massage element arrangement to the area.

2. The device as claimed in claim 1, wherein the laser diode arrangement comprises 2 laser InGaAlP diodes delivering light in the range of 600-700 nm.

3. The device as claimed in claim 1, wherein the light emitting diode arrangement comprises 4 light emitting diodes.

4. The device as claimed in claim 2, wherein the light emitting diode arrangement comprises 4 light emitting diodes.

5. The device as claimed in claim 1, wherein the device is powered by a rechargeable battery.

6. The device as claimed in claim 2, wherein the device is powered by a rechargeable battery.

7. The device as claimed in claim 3, wherein the device is powered by a rechargeable battery.

8. The device as claimed in claim 4, wherein the device is powered by a rechargeable battery.

9. A method of therapeutic treatment comprising the steps of:
an operator activating one or more of the laser diode arrangement, the light emitting diode arrangement and the massage element arrangement of the therapeutic device as claimed in claim 1 and;
directing at an area of the operator's body to permit the imparting of the energy created by the device to the area.

10. The method of claim 9, wherein said therapeutic device is the therapeutic device as claimed in claim 2.

11. The method of claim 9, wherein said therapeutic device is the therapeutic device as claimed in claim 3.

12. The method of claim 9, wherein said therapeutic device is the therapeutic device as claimed in claim 4.

13. The method of claim 9, wherein said therapeutic device is the therapeutic device as claimed in claim 5.

14. The method of claim 9, wherein said therapeutic device is the therapeutic device as claimed in claim 6.

15. The method of claim 9, wherein said therapeutic device is the therapeutic device as claimed in claim 7.

16. The method of claim 9, wherein said therapeutic device is the therapeutic device as claimed in claim 8.
